(19) United States
(12) Patent Application Publication
(10) Pub. No.: US 2022/0211440 A1
(43) Pub. Date: Jul. 7, 2022

(54) Camera-Assisted Image-Guided Medical Intervention

(71) Applicant: Siemens Healthcare GmbH, Erlangen (DE)

(72) Inventors: Thomas O'Donnell, New York, NY (US); Randolph M. Setser, Cornelius, NC (US)

(21) Appl. No.: 17/248,028

(22) Filed: Jan. 6, 2021

(51) Int. Cl. A61B 34/20; A61B 34/00; A61B 90/00

(52) U.S. Cl. CPC A61B 34/20; A61B 34/25; A61B 2090/062; A61B 90/37; A61B 2090/3762; A61B 90/361

(57) ABSTRACT

For camera-assisted, image-guided medical intervention, a camera is used to allow interaction for insertion point designation on the patient. The x-ray imager is used for guiding the intervention, but less radiation may be needed since the camera is used to assist in trajectory selection before the intervention and/or in guidance during the intervention.

### BACKGROUND

The present embodiments relate to image-guided medical intervention. During image-guided, percutaneous needle procedures using an angiography system, positioning the C-arm is not, in general, an interactive process. A three-dimensional (3D) radiologic image volume is acquired, either as a pre-procedural computed tomography (CT) or magnetic resonance (MR) scan or an intraprocedural cone beam CT (CBCT). If a pre-procedural scan is used, registration is performed. Using needle guidance software, the user then specifies a needle path from skin entry point to target using this image volume. The angiography system is driven to a bulls-eye position and the needle inserted into patient at the specified skin entry point. The needle insertion is monitored using specified progression views from the angiography system until the target is reached. This results in additional radiation to the patient and requires frequent delays while the interventionalist steps away from the patient hygienic area to avoid x-ray exposure.

### SUMMARY

Systems, methods, and computer readable media with stored instructions are provided for camera-assisted, image-guided medical intervention. One or more cameras are used to allow interaction for insertion point designation on the patient. The x-ray imager or imaging system is used for guiding the intervention, but less radiation may be needed since the camera is used to assist in trajectory selection before the intervention and/or in guidance during the intervention.

In a first aspect, a method is provided for camera-assisted, image-guided medical intervention. A camera captures an outer surface of a patient. The outer surface is registered with a 3D radiological scan. The camera captures an indicator manually controlled by an interventionalist, where the indicator is captured relative to the outer surface of the patient. An x-ray imager is positioned relative to the patient based on the indicator. The medical intervention is guided with the x-ray imager as positioned.

In one embodiment, the outer surface is registered with a CT scan as the radiological scan. The registration may be updated as the patient moves. In one embodiment, the camera is a depth camera positioned in a medical suite.

For some trajectory selection embodiments prior to puncture or intervention, the capture of the indicator is repeated as the interventionalist moves the indicator. The indicator is a wand held by the interventionalist in some embodiments, but other devices or even the interventionalist (e.g., finger or hand) may be used. The capture may be to determine a point of contact of the indicator with the patient and/or an angle of the indicator. The x-ray imager is positioned based on the point of contact and/or the angle. In one embodiment, during subsequent guidance (intraprocedural), x-ray images are acquired during needle-based intervention with a needle entering the patient at the point of contact and at the angle.

In further embodiments for trajectory selection, a representation of the indicator is displayed relative to the outer surface and/or an interior representation of the patient as the interventionalist manually positions the indicator. Once the desired entry point and/or angle of the indicator is selected, the system receives the acceptance of a current position and/or angle of the indicator. The current or subsequent capture of the indicator is performed for the positioning of the x-ray imager in response to the acceptance.

For some embodiments in guidance after puncture or during intervention, the camera captures a needle entering the patient. A depth of the needle within the patient is determined from the capturing by the camera. In other embodiments, the guidance of the needle (e.g., angle) is based on the camera captures. The x-ray system as positioned is used to confirm the guidance.

In a second aspect, a medical imaging system is provided for intervention guidance. A depth sensor is configured to measure depths to a patient (distance relative to a patient surface) and to sense a wand held relative to the patient. An image processor is configured to determine a point of entry into the patient based on the sensing of the wand by the depth sensor and to position a C-arm x-ray system relative to the determined point of entry. A display is configured to display a representation of the wand relative to the patient.

In one embodiment, the image processor is configured to register a radiological scan to the depths, and the display is configured to display the representation of the wand relative to an interior of the patient from the radiological scan.

In another embodiment, the image processor is configured to determine an angle at the point of entry based on the sensing of the wand and to position the C-arm x-ray system relative to the determined point of entry and the angle.

In yet another embodiment, the image processor is configured to determine a depth of a needle in the patient from data captured by the depth sensor, and the display is configured to display a representation of the needle within the patient at the depth.

In other embodiments, the image processor is configured to register the depth sensor, the patient, and the C-arm x-ray system using the depths. In some embodiments, the display is configured to display the representation as the wand moves relative to the patient, and the image processor is configured to set the point of entry in response to input on a user input.

In a third aspect, a method is provided for camera-assisted, image-guided medical intervention. A point of entry and angle of entry of a needle to a patient in a first image of interaction of a pointer with a patient is determined. A needle path for the needle within the patient is confirmed from the point of entry and at the angle with an x-ray imager positioned relative to the patient based on the point of entry and the angle.

In one embodiment, a manually positioned wand in the first image is used to determine the point and angle. In another embodiment, a depth of the needle in the patient is obtained from a second image.

Any one or more of the aspects described above may be used alone or in combination. These and other aspects, features and advantages will become apparent from the following detailed description of preferred embodiments, which is to be read in connection with the accompanying drawings. The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the embodiments. Moreover, in the figures, like reference numerals designate corresponding parts throughout the different views.

FIG. 1 is a flow chart diagram of one embodiment of a method for camera-assisted, image-guided medical intervention;

FIG. 2 is an example depth image;

FIG. 3 illustrates example displays for point of entry and/or angle representations;

FIG. 4 is a block diagram of one embodiment of a system for camera-assisted, image-guided medical intervention.

### DETAILED DESCRIPTION OF EMBODIMENTS

One or more 3D cameras (e.g., color and depth) is employed in the interventional suite for associating the patient's body to the 3D radiologic image volume interactively or otherwise. The placement of a 3D camera in the interventional suite has the potential to reduce radiation exposure and decrease the time for planning and/or the intervention. The camera images are used to assist in locating the point of entry and/or to guide the intervention, reducing the number of and time to acquire needed x-ray exposures.

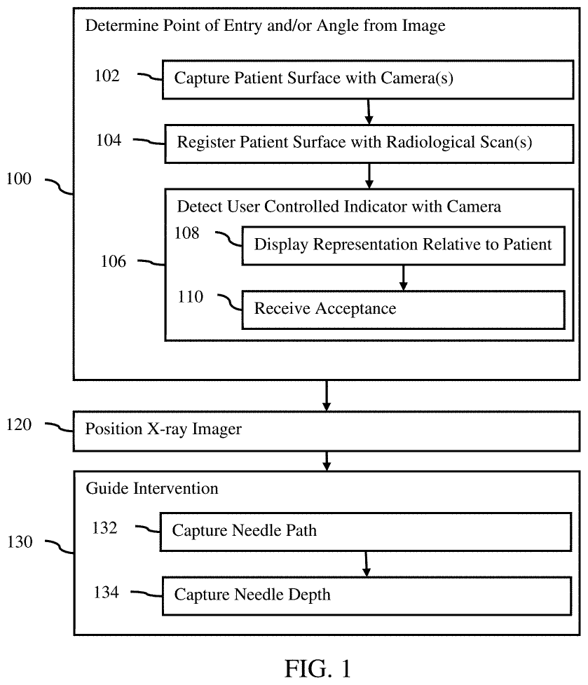

FIG. 1 is a flow chart diagram of one embodiment of a method for camera-assisted, image-guided medical intervention. A camera is used to interactively select the point of entry and/or angle of entry for an intervention while the patient is positioned in the intervention suite. The camera may be used to guide the intervention, such as determining a depth of the needle in the patient.

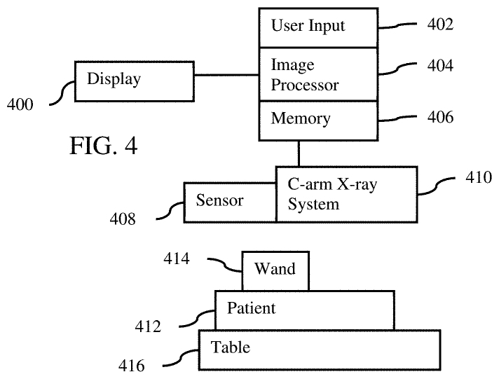

The method of FIG. 1 is performed by a medical imaging system, such as an image processor, camera, and/or x-ray imager. The camera captures the patient surface and/or an indicator positioned relative to the patient. An image processor determines the point of entry, angle, and/or depth using the captured surface data. The image processor positions the x-ray imager relative to the patient for guidance. The position is based on the selected point of entry and/or angle. In one embodiment, the system of FIG. 4 performs the method, but other systems may be used.

Additional, different or fewer acts may be provided. For example, acts 102 and/or 104 are not performed. As another example, the indicator is captured without the display of act 108. In another example, the x-ray imager is positioned in act 120 using other criteria than the selected trajectory. Act 100 may be performed without act 130, or act 130 may be performed without act 100. Other acts than acts 102-106 may be performed for act 100. Other acts than acts 132 and 134 may be performed for act 130.

The acts are performed in the order shown (top to bottom or numerical) or another order. For example, acts 102 and 104 are performed interleaved with or simultaneously with act 106. As another example, acts 132 and 134 are performed together or in an opposite order.

In act 100, an image processor determines a point of entry and/or angle of entry for an intervention. Where the intervention uses a needle, such as for a needle biopsy or extraction, the point of entry and/or angle of the needle relative to the patient is determined.

The determination is part of an interaction. The image processor uses input from an interventionalist, such as a physician, to determine. The input is from a camera, such as a camera capturing an interventionalist positioned indicator. None, one, or more additional sources, such as a user input device, may be used with the camera to determine.

The determination is performed prior to the intervention, such as prior to puncturing the skin of the patient. The intervention may be pre-planned, such as using a pre-operative 3D CT or MR scan of the patient and planning the point of entry, angle, depth, and/or another characteristic of the trajectory of the needle. The trajectory is designed to avoid puncturing any intervening organs. Once the intervention is to occur, the trajectory may be adjusted and/or it may be difficult to relate the selected trajectory from the pre-operative scan with an actual trajectory in the patient as the patient moves or has a different orientation.

The camera is used to determine the point of entry, angle, and/or depth on the actual patient. The patient is placed on a bed for the operation or intervention. The camera is used to input user selection and/or confirmation of the point of entry and angle on the patient as currently positioned. The camera allows for update of the point of entry and angle relative to the patient as the patient moves. During the intervention, the camera may be used to adjust the angle, update patient position, and/or determine the depth.

The camera captures one or more images (e.g., sets of surface data), such as images of an on-going or periodic video stream. One or more images represent an indicator for the point of entry, angle, and/or needle. The images capture the interaction of a pointer with the patient. Using the 3D camera, it is possible to interactively point to a position on the patient's body and see the corresponding point in the 3D image volume for trajectory selection.

Acts 102-106 show one example embodiment for determining the point of entry and/or angle from one or more images. Additional, different, or fewer acts may be provided.

In act 102, a camera captures an outer surface of a patient as an image. The camera captures the image as a two-dimensional distribution of pixels. Depth information may be captured as part of the image as well. The camera capture is performed in addition to any 3D “radiologic” scan (e.g., angiograph (dynaCT) or pre-acquired conventional CT) of the region or patient.

In one embodiment, the camera is a depth sensor, such as a 2.5D or RGBD sensor (e.g., Microsoft Kinect 2 or ASUS Xtion Pro). The depth sensor may directly measure depths, such as using time-of-flight, interferometry, or coded aperture. The depth sensor may be a camera or cameras capturing a grid projected onto the patient. The sensor may be multiple cameras capturing 2D images from different directions, allowing reconstruction of the outer surface from multiple images without transmission of structured light. Other optical or non-ionizing sensors may be used.

The sensor is directed at a patient. The sensor is positioned on a wall, ceiling, or elsewhere in the intervention suite or operating room, such as on a boom generally above the patient. The sensor captures the outer surface of the patient from one or more perspectives. Any portion of the outer surface may be captured, such as the entire patient viewed from one side from head to toe and hand to hand or just the torso. The sensor captures the outer surface with the patient in a particular position, such as capturing a front facing surface as the patient lies in a bed or on a table for treatment or imaging.

The outer surface is the skin of the patient. In other embodiments, the outer surface includes clothing. The sensor may use a frequency that passes through clothing and detects skin surface. Alternatively, the outer surface is the clothing.

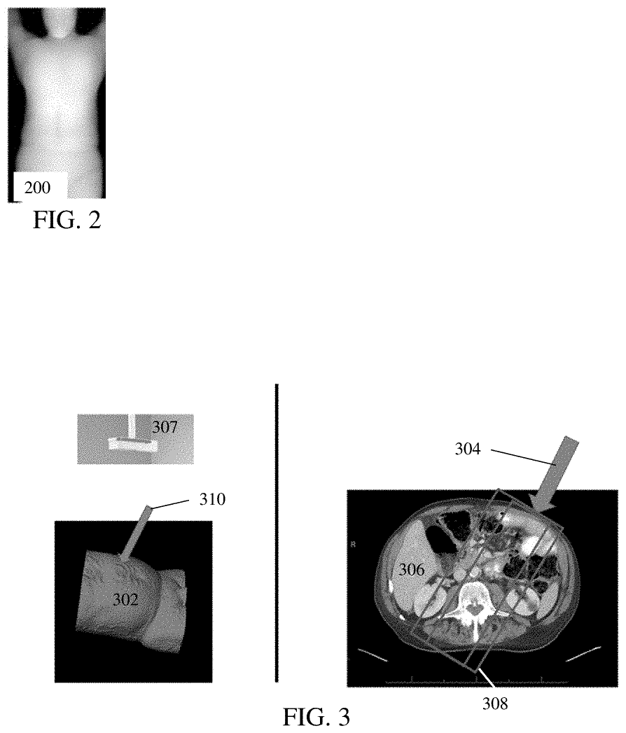

The outer surface is captured as depths from the sensor to different locations on the patient, a photograph of the outside of the patient, or both. The sensor outputs the sensed pixels and/or depths. The measurements of the outer surface from the sensor are surface data for the patient. FIG. 2 shows an example image 200 from surface data where the intensity in grayscale is mapped to the sensed depth. Alternatively, the sensor measurements are processed to determine the outer surface information, such as stereoscopically determining the outer surface from camera images from different angles with image processing.

The surface data is used at the resolution of the sensor. For example, the surface data is at 256×256 pixels. Other sizes may be used, including rectangular fields of view. The surface data may be filtered and/or processed. For example, the surface data is altered to a given resolution. As another example, the surface data is down sampled, such as reducing 256×256 to 64×64 pixels. Each pixel may represent any area, such as each pixel as down sampled to 64×64 representing 1 cm² or greater. Alternatively, the sensor captures at this lower resolution. The surface data may be cropped, such as limiting the field of view. Both cropping and down sampling may be used together, such as to create 64×64 channel data from 256×312 or other input channel data.

In another approach, the surface data is normalized prior to input. The surface data is rescaled, resized, warped, or shifted (e.g., interpolation). The surface data may be filtered, such as low pass filtered.

In act 104, the image processor registers the outer surface with a radiological scan. For example, the outer surface is registered with a CT, MR, or other radiological scan. The radiological scan may be a pre-operative scan of the patient. The patient is scanned in 3D prior to being placed in the intervention suite for the intervention. In other embodiments, the radiological scan occurs once the patient is positioned in the intervention suite, such as after the patient is positioned on the bed for the intervention. A C-arm x-ray scan is performed in a CT-like scan to scan a volume of the patient.

The outer surface is represented in both the camera image and the radiological scan. By performing a rigid or non-rigid correlation or fitting, the same locations on the outer surfaces of the radiological scan and the camera capture are matched or identified. A deformable mesh or model may be used to relate the spatial positions of the two data sets to each other.

In one embodiment, the 3D surfaces are fit together, such as with an optimization. In other embodiments, fiducials detectable to both the camera and the radiology system of the radiological scan are positioned on the patient. The fiducials are detected and used to register. The needle or another intervention device may be placed against or partially in the patient and represented in both types of imaging, so may be used for registration.

The registration aligns the coordinate system of the camera with the radiological scanner used to perform the radiological scan. The registration also relates the position of the patient to the camera and/or radiological scan. The registration may be repeated as the patient moves. The update adjusts for any patient movement. Since the camera may capture images periodically or in a video stream, the patient position and corresponding registration may be repetitively performed and updated.

In act 106, the image processor detects a manually positioned indicator in one or more photographs from the camera. The image processor applies image processing, such as fitting a model and/or applying a machine-learned detector, to find the indicator in the photograph. The depth data and/or photograph are used to detect the indicator having one or more known characteristics.

In one embodiment, the indicator is a wand, such as a rod with color coding and/or a pattern. The needle or another device to be used in the intervention may be used as the indicator. The image processor detects the wand. The wand may be a pen, pencil, or marker with or without color coding or a pattern. Other devices than a wand may be used, such as any pointer. In other embodiments, a beam or laser is used. The camera either senses the beam or the projection from the beam on the patient. The physician or interventionalist body part may be used as the indicator, such as tip of a finger, finger, or arm.

The indicator is manually positioned by the user. For example, the user holds the indicator adjacent to the patient. As another example, the user places the indicator on the patient and releases the indicator. In yet another example, the user places the indicator with a mechanical arm.

The indicator is placed relative to the outer surface of the patient. For example, the indicator is positioned to contact the patient's outer surface. In other embodiments, the orientation and position of the indicator spaced from the patient are used to find an intersection with the patient.

The placement and/or orientation of the indicator indicate the point of entry and/or angle for intervention guidance. The camera captures the indicator manually controlled by an interventionalist. For example, the depth camera positioned in the medical suite captures an image of the patient and the indicator as held by the user. The detected indicator has a position relative to the outer surface of the patient. The position is a point or line, such as detecting the point of contact of the indicator with the patient or detecting the point of contact and an orientation about that point. The position may be an intersection of a virtual line through the indicator, indicating the point of entry for intervention by the intersection of the virtual line along the indicator with the outer surface of the patient.

The point of entry and/or angle are determined from the point of contact and/or point of intersection and/or orientation of the indicator. The depth may be determined, such as the indicator including a marking for depth that may be set by the user on the indicator.

Acts 108 and 110 are example acts used to select the trajectory using detection of the indicator. Other acts may be used.

In act 108, the image processor generates and causes display on the display of a representation of the indicator relative to the outer surface and/or an interior representation of the patient. FIG. 3 shows one or two examples. Other examples showing a representation of the detected indicator relative to the patient may be used.

The left side of FIG. 3 shows an outer surface 302 of the patient and an arrow-shaped indicator 310 placed against the patient. While the left side is described as repenting real-life or an actual occurrence, this image may be displayed as one example of a representation displayed to the user to assist in placement. In e this example, an outer surface of the patient 302 is displayed as a 3D rendering. A representation 304 of the indicator showing an orientation and entry position relative to the outer surface 302 is rendered with the surface 302 or overlaid as a graphic on the surface 302. The image of the surface 302 is generated to show the point of entry on the surface and/or the angle of entry detected from the position of the indicator89.

In another example of a display representation, the image is of an interior 306 of the patient, such as from the radiological scan. The representation 304 of the indicator is shown as an arrow representation relative to the interior 306. Other indicator representations, such as a trajectory, may be used. The indictor may be represented to extend into or through the interior 306. One or more box outlines 308 may be represented, such as for forming multi-planar reconstruction (MPR) images of two orthogonal planes with an intersection formed by a virtual line from the indictor 304. The angle of the indicator is used to position the planes of the MPR. The image or images (e.g., 3D rendering and/or MPR images) of the interior 306 are generated to show the point of entry on the surface and/or the angle of entry.

The penetrating depth may be shown. The detected depth from the indicator is used to show the depth within the patient. The angle of approach and penetrating depth of the designated instrument is visualized directly on the radiologic 3D scan. MPR images and/or volume rendering are created to show the point of entry, angle, and depth.

As the interventionalist manually positions the indicator or upon triggering by the interventionalist, the representation of the indictor 304 relative to the patient is displayed. The interventionalist may interactively touch any portion of the patient visible to the camera 307 with a designated instrument (i.e., indicator) and see the corresponding point in the radiologic scan or camera image. The capture of images of the indicator relative to the patient is repeated as the interventionalist moves the indicator. The image processor, using images captured by the camera 307, tracks the indicator.

In act 110, the image processor receives acceptance of a current position, angle, and/or depth of the indicator. By moving the indicator relative to the patient, different entry points, angles, and/or depths are displayed relative to the patient in act 108. Each represents at least parts of different trajectories. The user moves the indicator until a desired trajectory, such as one created in pre-planning, is found. Upon finding the desired trajectory, the user indicates acceptance. When the interventionalist is happy with the position, angle, and/or depth of approach, the acceptance is communicated to the C-arm which automatically moves into place based on the registration.

The acceptance is communicated by entry on a user input device. For example, a key on a keyboard or button on a mouse is depressed to indicate acceptance. As another example, the indicator includes a transmitter and user input (e.g., button). The user activates the user input on the indicator to indicate acceptance. In another example, a hand motion, another visual input, or voice control is provided. The image processor detects the acceptance by image processing of the image or video captured by the camera.

The current detected point of entry, angle, and/or depth is used upon receipt of acceptance. Alternatively, the camera captures a depth image (RGBD) upon receipt of the acceptance. The point of entry, angle, and/or depth detected in the newly captured image is used. The point, angle, and/or depth as an average for multiple images over a given period may be used. By using either the current camera image or a subsequently triggered camera image, the capture of the indicator is performed in response to the acceptance.

In act 120, the image processor controls positioning of an x-ray or other imager. The imager is positioned relative to the patient. The imager is positioned to image an interior of the patient in a way that will show the intervention device (e.g., needle) within the patient to confirm following of the desired trajectory. For example, the x-ray images are positioned to capture MPRs or orthogonal radiographs along the trajectory. A C-arm is positioned to allow for translation and/or rotation to image a volume of the patient about the trajectory.

The imager is positioned relative to the patient based on the indicator. The point of entry, angle, and/or depth are used to position the imager relative to the patient. For example, a C-arm positions an x-ray source and detector relative to the selected trajectory. The positioning occurs automatically in response to acceptance of act 110 or other selection of the trajectory.

The images from the x-ray imager and the camera may be registered to display an updated view of the interior 306 relative to the indicator 304. The registration between 3D camera and x-ray imager could be automated since part of the x-ray imagers may be captured and detected in an image of the camera.

The trajectory selection may be updated. Act 100 may be repeated during the intervention, such as to adjust a depth and/or angle. The needle may be used as the indicator. Act 120 is likewise repeated to align the x-ray imager with the updated trajectory.

In act 130, the image processor uses the camera and/or x-ray imager to guide the medical intervention. For example, a needle is used in the intervention. The needle is to puncture the skin of the patient and travel within the patient to a point or region. The trajectory is set to avoid puncturing one or more organs along the trajectory. A straight or slightly curved trajectory through the patient is planned. Once the intervention is to begin, the point of entry on the patient as positioned on the bed in the intervention suite and the angle of the needle is established, such as through the processor performing act 100. Continued imaging is used to confirm the proper placement. Continued imaging is used to confirm that the needle is following the trajectory within the patient as the needle is inserted.

The camera may be used to provide guidance for initial puncture. The camera may be used to provide guidance after puncture, such as by establishing angle of the needle relative to the patient and/or depth of needle entry into the patient. The x-ray imager may be used to provide guidance, such as after puncture. The needle is detected from projection or volume scanning of the patient and needle. Alternatively, the needle may be viewed in MPR or volume rendered images. The resulting images are displayed to confirm that the needle is following the desired trajectory in the patient. By including the information from the camera, fewer x-ray images may be needed, reducing radiation dose and time spent in the intervention.

The medical intervention is guided, at least in part, by the x-ray imager as positioned. The positioning of the x-ray imager to align an axis between the source and detector to be the same as the intervention trajectory or at a desired offset and/or angle to the trajectory (e.g., orthogonal) results in x-ray images or a scan volume more likely to show the needle relative to organs of interest (e.g., organs to be avoided and/or target organ).

The point of entry and/or the angle from act 100 are used to position the x-ray imager in act 120 for confirming the needle path or trajectory within the patient. Prior to and/or after puncture, x-ray imaging may be used to confirm proper placement of the needle and/or trajectory. The positioning of the x-ray imager in act 120 relative to the patient assists in confirming the proper needle position and/or path. X-ray images prior to and/or during needle-based intervention with a needle entering the patient at the point of contact and at the angle are acquired and displayed.

Acts 132 and 134 represent two acts for guiding the intervention using, at least in part, the camera. Additional, different, or fewer acts may be used.

In act 132, the camera captures a needle entering the patient. One or more camera images capture the interventionalist inserting the needle into the patient. The angle and/or point of entry of the needle are determined from the images. This angle and/or point of entry may be used to represent the current trajectory of the needle within the patient. An image or images from a pre-operative scan and/or a most recent x-ray scan are used to show the needle and/or the trajectory (i.e., where the needle is going to progress) based on the camera detected needle. The point of entry and/or angle from the camera assist in determining the needle location and/or expected trajectory from the current location, guiding the interventionalist in moving the needle. The camera may be relied on to show the path in the patient based on the needle portion outside the patient. The x-ray imager as positioned relative to the current trajectory from the camera may be used less frequently than the camera to confirm the position of the needle and/or trajectory within the patient.

In act 134, the camera captures a depth of the needle within that patient. The length of the needle outside of the patient or extending from the patient's skin is determined. For example, a machine-learned detector and/or markings on the needle are used to identify the needle, including the point of entry, end outside of the patient, and orientation. A distance or length of the needle outside the patient is subtracted from a known length of the needle. The result calculated by the image processor is the depth of the needle within the patient. Different RGBD images are captured at different times (e.g., video) to provide the depth of the needle in the patient over time, such as while the interventionalist performs the intervention.

The pre-operative radiological scan and/or a most recent scan by the x-ray imager may be used with the camera-based measurement of depth to indicate the location of the needle along the trajectory. One or more images representing the interior of the patient and the location of the needle and/or tip of the needle show the extent of the needle within the patient. The pre-operative radiological scan and/or a most recent x-ray scan are used to generate one or more images showing the needle (e.g., highlight the needle) and/or showing a representation of the needle. In one embodiment, the depth is used to detect the needle tip in a most recent scan by the x-ray imager. The detected tip may be highlighted in one or more images displayed to the user. The displayed needle within the interior of the patient is used to guide the intervention, allowing the interventionalist to move the needle deeper and/or engage with the tissue of interest.

FIG. 4 shows one embodiment of a medical imaging system for intervention guidance. The medical imaging system includes the display 400, user input 402, memory 406, and image processor 404. The medical imaging system also includes the sensor 408 for sensing (imaging) an outer surface of a patient and/or a wand. The display 400, image processor 404, and memory 406 may be part of the C-arm x-ray system 410, a computer, server, workstation, or another system for image processing medical images from a scan of a patient. A workstation or computer with the camera and without the C-arm x-ray system 410 may be used as the medical imaging system.

Additional, different, or fewer components may be provided. For example, a computer network is included for remote image generation of locally captured surface data or for local imaging from remotely captured surface data. As another example, one or more machine-learned detectors or classifiers are applied to locate a needle in an x-ray image, to position the C-arm X-ray system based on trajectory, to locate the wand from an image, and/or to register scan data from different imagers (e.g., the sensor 408 and the C-arm X-ray system 410).

The sensor 408 is a depth sensor or camera. LIDAR, 2.5D, RGBD, stereoscopic optical sensor, or other depth sensor may be used. Alternatively, a camera without depth sensing is used. One sensor 408 is shown, but multiple sensors may be used, such as viewing the patient 412 on the bed or table 416 from different angles, and/or distances. A light projector may be provided. The sensor 408 may directly measure depth from the sensor 408 to the patient and/or indicator (e.g., wand). The sensor 408 may include a separate processor for determining depth measurements from images and/or detecting objects represented in images, or the image processor 404 determines the depth measurements from images captured by the sensor 408. The depth may be relative to the sensor 408 and/or a bed or table 416.

The sensor 408 is directed to the patient 412. The sensor 408 may be part of or connected to the C-arm x-ray system 410 or is separate from the C-arm x-ray system 410. In one embodiment, one or more sensors 408 are positioned on the ceiling and/or walls of the intervention suite.

The sensor 408 is configured to measure depths to or from a patient, needle, and/or a wand 414. The depths are distances from the sensor 408, table 416, or other location to the patient and/or wand at various locations on the patient and/or wand. Any sample pattern over the patient, needle, and/or wand may be used. The sensor 408 outputs depth measurements and/or a surface photograph.

In one embodiment, the wand 414 includes a marker, such as a marker on a ball at an end of the wand 414 to be held away from the patient 412. The wand 414 may fit over, along, or against a needle guide, such as a pivotable needle guide stuck or pasted to the patient. The needle guide may include a target or detectable pattern to designate the desired or selected entry point. The needle guide and wand 414 are moved relative to the patient to find the desired entry point, then the needle guide is pasted to the patient. Since the needle guide may be rotatable but generally stiff, the wand 414 as mated with the needle guide may be moved to establish the angle and then held in place by the needle guide to confirm the point of entry and angle.

The C-arm x-ray system 410 is an x-ray imager, such as an angiography system. The C-arm x-ray system 410 operates pursuant to one or more settings to position and operate C-arm (e.g., gantry), an x-ray source connected to the C-arm, and detector connected to the C-arm. The settings control the location or region of the patient being scanned and the scan sequence. For example, a CT type or CT-like scan is performed by moving the C-arm relative to the patient. The medical scanner is configured to generate diagnostic image information. The configuration uses settings for one or more parameters, such as an X-ray source voltage, table position and/or range of movement, gantry position and/or range of movement, focus, field of view, scan density, detector thresholds, transmission sequence, image processing settings, filtering settings, or image generation settings. The patient 412 is imaged by the medical scanner using the settings. In alternative embodiments, another type of medical scanner is configured to scan an internal region of the patient 412 and generate diagnostic information from the scan. The medical scanner may be a CT, MR, PET, SPECT, X-ray, or ultrasound scanner.

The user input 402 is configured, through a user interface operated by the image processor 404 or another processor, to receive and process user input. For example, acceptance of a currently designated or detected entry point and/or angle is received by the user input 402. The user input 402 is a device, such as keyboard, button, slider, dial, trackball, mouse, or another device).

The image processor 404 is a control processor, general processor, digital signal processor, three-dimensional data processor, graphics processing unit, application specific integrated circuit, field programmable gate array, artificial intelligence processor, digital circuit, analog circuit, combinations thereof, or another now known or later developed device for image processing and/or intervention guidance. The image processor 404 is a single device, a plurality of devices, or a network. For more than one device, parallel or sequential division of processing may be used. Different devices making up the image processor 404 may perform different functions, such as detecting a wand from depth data by one device and generating an image or images representing a trajectory and/or needle relative to the patient by another device. In one embodiment, the image processor 404 is a control processor or other processor of a C-arm x-ray system 410. The image processor 404 operates pursuant to and is configured by stored instructions, hardware, and/or firmware to perform various acts described herein.

The image processor 404 is configured to register a radiological scan to the camera image (e.g., surface formed by the depths). The surface data (e.g., image including pixels in gray scale or color and/or depth information) is registered with a pre-operative or an interventional (during the intervention) radiological scan. The registration uses the outer surface of the patient (e.g., depth measurements), which is common to the different imagers. Thus, the depth sensor 408, patient, and C-arm X-ray system 410 are registered so that a location in one coordinate system is determinable in another coordinate system. The registration may be updated, such as to account for patient movement.

The image processor 404 is configured to determine a point of entry into the patient 412 based on the sensing of the wand 414 by the depth sensor 408. This determination allows setting of the point of entry. The point of entry is set in response to input on the user input 402. Once the user selects a desired trajectory relative to the patient 412 on the table 416, the user input 402 is operated to confirm selection.

The image processor 404 is configured to determine an angle at the point of entry based on the sensing of the wand 414. The surface data from the depth sensor 408 is used to detect the wand 414, including an orientation of the wand 414. The orientation of the wand 414 relative to the sensor 408 and the patient 412 is used to determine an angle of the trajectory in the patient.

The image processor 404 is configured to position the C-arm X-ray system 410. The C-arm is moved to orient the X-ray source and detector relative to the selected trajectory given the current position of the patient 412. The C-arm X-ray system is positioned relative to the point of entry and/or the angle. The trajectory path, as positioned on the patient 412, is used to position the C-arm for CT-like or type of imaging by rotating and/or translating the source and detector about the patient. The positioning of the C-arm may include moving the bed or table 416.

The image processor 410 is configured to guide the intervention. Images are generated for selecting the point of entry and/or angle, such as the right image of FIG. 3 showing the representation 304 of the wand 414 or trajectory relative to the outer surface 302 or interior 306 of the patient 412. Images are generated during the intervention for showing the current position of the needle and/or the projected trajectory of the needle. The images may be of the interior of the patient. The image processor 410 may be configured to determine a depth of a needle in the patient 412 from data captured by the depth sensor 408. The depth may be reflected in the image, such as by highlighting a needle tip. Alternatively or additionally, the depth is used to position the C-arm for scanning.

The display 400 is a CRT, LCD, projector, plasma, printer, tablet, smart phone, or another now known or later developed display device for displaying the trajectory and/or needle, such as an image of the interior or exterior of the patient 412 including the trajectory or needle. The display 400 displays a medical image of the patient and/or of the trajectory. In one embodiment, the display 400 is configured by the image processor 404 to display a representation of the wand 414 relative to the patient 412 for selecting the trajectory relative to the current position of the patient 412. The representation of the wand is displayed relative to an interior of the patient 412 from a radiological scan and/or the exterior of the patient from the sensor 408. In another embodiment, the display 400 is configured to display the representation of the trajectory as the wand 414 moves relative to the patient 412 for selecting one of various different possible trajectories. In other embodiments, the display 400 is configured to display a representation of the needle within the patient 412, such as showing the depth of the needle determined at least in part from the surface data of the sensor 408.

The sensor measurements, surface data, trajectory, images, scan data, and/or other information are stored in a non-transitory computer readable memory, such as the memory 406. The memory 406 is an external storage device, RAM, ROM, database, and/or a local memory (e.g., solid state drive or hard drive). The same or different non-transitory computer readable media may be used for the instructions and other data. The memory 406 may be implemented using a database management system (DBMS) and residing on a memory, such as a hard disk, RAM, or removable media. Alternatively, the memory 406 is internal to the processor 404 (e.g. cache).

The instructions for implementing the methods, processes, and/or techniques discussed herein are provided on non-transitory computer-readable storage media or memories, such as a cache, buffer, RAM, removable media, hard drive or other computer readable storage media (e.g., the memory 406). Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination.

In one embodiment, the instructions are stored on a removable media device for reading by local or remote systems. In other embodiments, the instructions are stored in a remote location for transfer through a computer network. In yet other embodiments, the instructions are stored within a given computer, CPU, GPU or system. Because some of the constituent system components and method steps depicted in the accompanying figures may be implemented in software, the actual connections between the system components (or the process steps) may differ depending upon the manner in which the present embodiments are programmed.

Various improvements described herein may be used together or separately. Although illustrative embodiments of the present invention have been described herein with reference to the accompanying drawings, it is to be understood that the invention is not limited to those precise embodiments, and that various other changes and modifications may be affected therein by one skilled in the art without departing from the scope or spirit of the invention.

What is claimed is:

1. A method for camera-assisted, image-guided medical intervention, the method comprising:
capturing, with a camera, an outer surface of a patient;
registering the outer surface with a radiological scan;
capturing, with the camera, an indicator manually controlled by an interventionalist, the indicator being relative to the outer surface of the patient;
positioning an x-ray imager relative to the patient based on the indicator; and
guiding the medical intervention with the x-ray imager as positioned.

2. The method of claim 1 wherein registering comprises registering the outer surface with a computed tomography scan as the radiological scan.

3. The method of claim 1 wherein registering comprises updating registration as the patient moves.

4. The method of claim 1 wherein capturing the indicator is repeated as the interventionalist moves the indicator.

5. The method of claim 1 wherein capturing the indicator comprises capturing a wand held by the interventionalist.

6. The method of claim 1 wherein capturing the indicator comprises capturing a point of contact of the indicator with the patient and an angle of the indicator, wherein positioning comprises positioning the x-ray imager based on the point of contact and the angle, and wherein guiding comprises acquiring x-ray images during needle-based intervention with a needle entering the patient at the point of contact and at the angle.

7. The method of claim 1 further comprising displaying a representation of the indicator relative to the outer surface and/or an interior representation of the patient as the interventionalist manually positions the indicator.

8. The method of claim 1 further comprising receiving acceptance of a current position and/or angle of the indicator, wherein capturing the indictor is performed for the positioning in response to the acceptance.

9. The method of claim 1 wherein capturing the outer surface comprises capturing with a depth camera positioned in a medical suite.

10. The method of claim 1 further comprising capturing, with the camera, a needle entering the patient, wherein guiding comprises determining a depth of the needle within the patient from the capturing by the camera.

11. The method of claim 1 further comprising capturing, by the camera, a needle entering the patient, wherein guiding comprises guiding the needle based on the capturing of the needle and confirming with the x-ray system as positioned.

12. A medical imaging system for intervention guidance, the medical imaging system comprising:
a depth sensor configured to measure depths to a patient and to sense a wand held relative to the patient;
a C-arm x-ray system;
an image processor configured to determine a point of entry into the patient based on the sensing of the wand by the depth sensor and to position the C-arm x-ray system relative to the determined point of entry; and
a display configured to display a representation of the wand relative to the patient.

13. The medical imaging system of claim 12 wherein the image processor is configured to register a radiological scan to the depths and wherein the display is configured to display the representation of the wand relative to an interior of the patient from the radiological scan.

14. The medical imaging system of claim 12 wherein the image processor is configured to determine an angle at the point of entry based on the sensing of the wand, wherein the image processor is configured to position the C-arm x-ray system relative to the determined point of entry and the angle.

15. The medical imaging system of claim 12 wherein the image processor is configured to determine a depth of a needle in the patient from data captured by the depth sensor, and wherein the display is configured to display a representation of the needle within the patient at the depth.

16. The medical imaging system of claim 12 wherein the image processor is configured to register the depth sensor, the patient, and the C-arm x-ray system using the depths.

17. The medical imaging system of claim 12 further comprising a user input, wherein the display is configured to display the representation as the wand moves relative to the patient, and wherein the image processor is configured to set the point of entry in response to input on the user input.

18. A method for camera-assisted, image-guided medical intervention, the method comprising:
determining a point of entry and angle of entry of a needle to a patient in a first image of interaction of a pointer with a patient; and
confirming a needle path for the needle within the patient from the point of entry and at the angle with an x-ray imager positioned relative to the patient based on the point of entry and the angle.

19. The method of claim 18 wherein determining comprises detecting a manually positioned wand in the first image.

20. The method of claim 18 further comprising obtaining a depth of the needle in the patient from a second image.
